(19) United States
(12) Patent Application Publication
(10) Pub. No.: US 2013/0268074 A1
(43) Pub. Date: Oct. 10, 2013

(54) PROSTHETIC MENISCI AND METHOD OF IMPLANTING IN THE HUMAN KNEE JOINT

(71) Applicant: Robert Walter Vowles, Romsey (AU)

(72) Inventor: Robert Walter Vowles, Romsey (AU)

(73) Assignee: PROSTHEXIS PTY LTD, Romsey, Victoria (AU)

(21) Appl. No.: 13/995,992

(22) PCT Filed: Dec. 21, 2011
(86) PCT No.: PCT/AU2011/001657

§ 371 Date: Jun. 20, 2013

(30) Foreign Application Priority Data
Dec. 21, 2010 (AU) 2010905580

(51) Int. Cl. A61F 2/38

(52) U.S. Cl. CPC A61F 2/3872
USPC 623/14.12

(57) ABSTRACT

Prosthetic knee menisci to be implanted in place of deteriorated native menisci to prevent damage to the articular cartilage of the femoral and tibial condyles and, thereby, to arrest the progressive development of osteoarthritis; said prosthetic menisci being made as a hollow form and inflated after implantation by injection of a settable polymer to shape them into congruence with the femoral and tibial condyles; being sized for the femoral and tibial condylar surfaces; having internal reinforcement for strength and durability; being made from materials having elastomeric characteristics similar to those of native menisci; having bearing surfaces treated chemically and/or physically to improve the efficiency of lubrication by synovial fluid and to enhance the wear characteristics of the bearing surfaces; and being restricted in translation within the interarticular space by anchorage of their anterior and posterior horns and by the provision of secondary locating elements.

This invention relates generally to surgical methods of alleviating the discomfort and impaired mobility resulting from deterioration or injury to the meniscus of the human knee. More specifically, it relates to a method of replacing either or both of the natural menisci in the human knee with prosthetic menisci.

Arthritic deterioration of the knee joint is now extremely common in the western world. A progressively aging population is doubtless a contributory factor. As a result of the limited options available, the number of total knee joint replacements being performed annually in the United States is rapidly becoming an unsustainable burden on the healthcare system. Worldwide, the number of procedures is increasing almost exponentially, despite the fact that it is irreversible and may ultimately require revision. Indeed, the number of total knee replacement revisions has become so great that it now constitutes a well defined sub-speciality in orthopaedic surgery. Clearly, any alternatives to forestall the need for total knee replacement should be considered.

The knee complex is one of the most frequently injured joints in the human body. The knee joint works in conjunction with the hip joint and ankle to support the weight of the body during static, erect posture. Dynamically, it is responsible for moving and supporting the body during a variety of both routine and difficult activities. The fact that the knee must fulfil both major stability and major mobility functions is reflected in its complex structure and functionality.

The two major bones of the leg are the femur, the proximal end of which pivots at the hip joint and the tibia, the distal end of which pivots at the ankle joint. The femur and tibia pivot are joined in an articulated relationship at the knee by the tibiofemoral joint, the largest in the body. The distal end of the femur and proximal end of the tibia are expanded and, although this provides some basis for stability, there is no great adaptation of the bony ends one to another. The distal end of the femur is developed into two discrete condyles, the lower surfaces of which are smoothly rounded and covered in (hyaline) articular cartilage which provides a smooth bearing surface. The anteroposterior convexity of the condyles is not consistently spherical, having a smaller radius of curvature posteriorly. Separated by the intercondylar notch, the condyles have considerable posterior development to accommodate flexing of the knee joint. The medial condyle has greater, posterior development and a greater vertical development which compensates for a degree of obliquity of the shaft of the femur. The lateral femoral condyle is shifted anteriorly in relation to the medial condyle and the articular surface of the lateral condyle is shorter than that of the medial condyle. The proximal end of the tibia comprises shallow, concave lateral and medial plateaus covered with articular cartilage, the medial plateau being larger than the lateral. The tibial plateaus are separated by the lateral and medial intercondylar eminences or tubercles. The femoral condyles are located by and pivotally supported in semi-annular fibro-cartilaginous structures, the menisci, located on the tibial plateaus. These accessory joint structures provide smooth, concave upper surfaces forming complementary bearing surfaces against which the condyles work during articulation of the knee. The knee is also supported by a laterally-located, long auxiliary bone, the fibula. The fibula is strongly bound to the tibia at its distal end, but has a small synovial joint at its upper end joined to the tibial epiphysis. The capsule of the superior tibiofibular joint is reinforced by anterior and posterior ligaments.

The patella (kneecap) is embedded in the quadriceps tendon which connects the quadriceps musculature of the anterior upper thigh to the patella, the patella being connected by the patellar ligament to the tibia just beneath the knee. In simple terms, the posterior surface of the patella is provided with a projection which, during knee flexion, is slidingly displaced in the trochlea, a groove formed in the anterior surface of the femur, between the condyles. The contact zones of patella and femur are covered with smooth articular cartilage providing low friction, complementary working surfaces. The combination of quadriceps tendon, patella and patellar ligament acts rather like a pulley, transmitting forces generated by the quadriceps musculature to the tibia via the flexed knee to straighten the leg or decelerate the rate of flexion. The patella obviously also serves the further function of protecting the knee joint from impact damage.

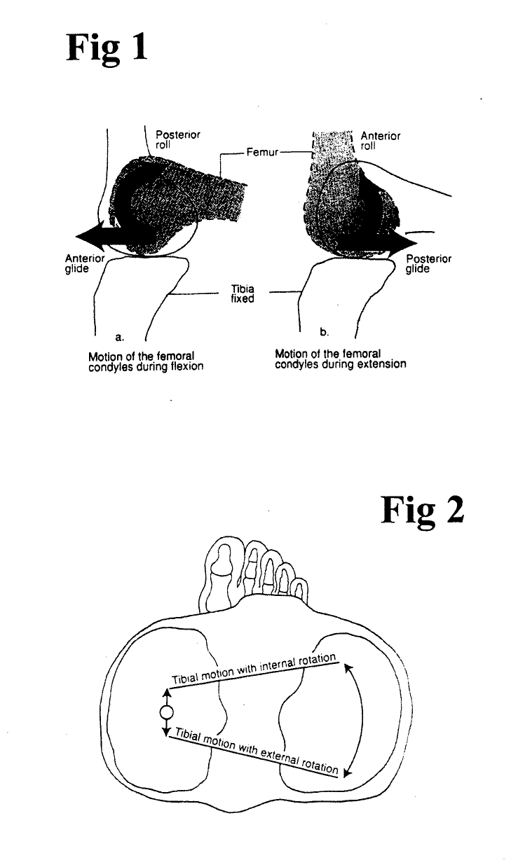

The large articular surface of the femur and the relatively small tibial condyle create a potential problem as the femur commences rotation on the tibia. During extended flexion, in order for them to remain on the tibial plateaus, the femoral condyles must simultaneously glide anteriorly and, during extension, simultaneously glide posteriorly. As stated, the cruciate ligaments act to substantially locate the condyles on the tibia during flexion and extension. As illustrated in a. of FIG. 1, during flexion of the knee joint, tension applied by the anterior cruciate ligament restrains the condyles from posterior displacement. Similarly, as illustrated in b. of FIG. 1, during extension of the knee joint, the posterior cruciate ligament restrains the condyles from anterior displacement. These effects are reinforced by the capsule and the layers of ligamentous and tendinous tissue surrounding the knee joint. For example, the iliotibial band, which transmits forces from thigh muscles to the tibia, provides lateral support to the knee joint and, during flexion, restricts excessive anterior translation of the tibia under the femur.

Medial and lateral rotation of the knee joint are angular motions that are named for the motion (or relative motion) of the tibia on the femur. These axial rotations of the knee joint occur about a longitudinal axis that runs through or close to the medial tibial intercondylar tubercle. Consequently, the medial condyles acts as pivot points while the lateral condyles move through a greater arc of motion, regardless of the direction of rotation. This is illustrated in FIG. 2. As the tibia laterally rotates on the femur, the medial tibial condyle moves only slightly anteriorly on the relatively fixed medial femoral condyle, whereas the lateral tibial condyle moves a larger distance posteriorly on the relatively fixed lateral femoral condyle. During tibial medial rotation, the medial tibial condyle moves only slightly posteriorly, whereas the lateral condyle moves anteriorly through a longer arc of motion. During both medial and lateral rotation, the knee joint menisci will distort in the direction of movement of the corresponding femoral condyle and, therefore, maintain their relationship to the femoral condyles as they did in flexion and extension. The range of knee joint rotation possible depends upon the flexion/extension position of the knee. When the knee is in full extension, the ligaments are taut, the tibial tubercles are lodged in the intercondylar notch and the menisci are tightly interposed between the articulating surfaces; consequently, little axial rotation is possible. As the knee flexes towards 90 degrees, capsular and ligamentous laxity increase, the tibial tubercles are no longer in the intercondylar notch, and the condyles of the tibia and femur are free to move in relation to each other. The maximum range of axial rotation is available at 90 degrees of knee flexion: the range of lateral rotation being 0 to 20 degrees and the range of medial rotation, 0 to 15 degrees, giving a total medial/lateral rotation of up to 35 degrees.

The knee menisci were once thought to be just a form of vestigial tissue but are now understood to be vital to the proper functioning of the knee joint. In addition to enhancing joint congruence, the menisci play an important role in distributing forces through the knee, in reducing friction between the femur and tibia and in absorbing shock loadings to the knee. The menisci cover between one half and two thirds of the tibial articular plateau and are open towards the tibial tubercles, the lateral meniscus covering a greater percentage of the smaller lateral tibial plateau. As a result of its larger exposed surface, the medial condyle has a greater susceptibility to the enormous compressive loads that pass through it during routine activities. Although compressive forces in the knee may reach one or two times body weight during gait and stair climbing and three to four time body weight during running, the menisci assume 50% to 70% of this imposed load. Meniscal motion on the tibia is limited by multiple attachments to surrounding structures, some common to both menisci and some unique to each. To accommodate deviations of the femoral condyles from sphericality, the menisci obviously possess some freedom of movement. The medial meniscus has greater ligamentous and capsular constraints, limiting its translation to a greater extent than that of the lateral meniscus. The relative lack of mobility of the medial meniscus may contribute to its greater incidence of injury—some nine times greater than that of the lateral meniscus.

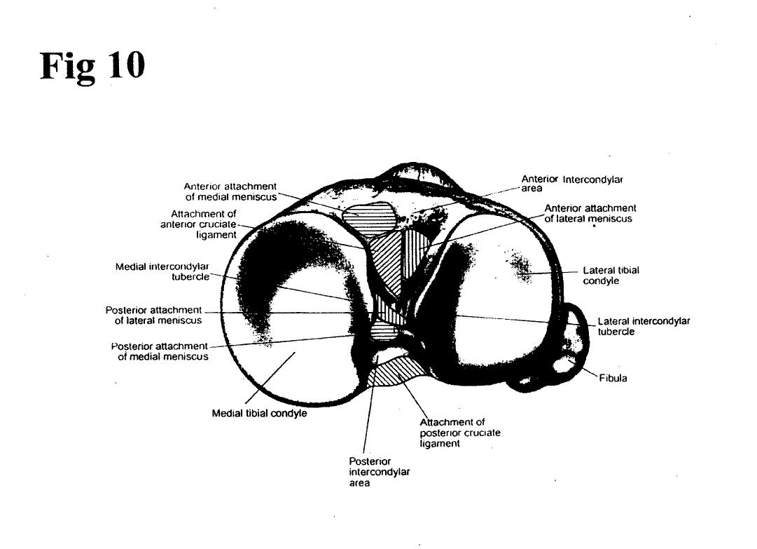

The menisci are best described as crescent-shaped wedges of fibrocartilage supported upon the peripheral aspects of the articular surfaces of the proximal tibia. They function to effectively deepen the medial and lateral tibial fossae for articulation with the condyles of the femur. They are thickest at their external margins and taper to thin, unattached edges as they extend radially inwards. The superior surfaces of the menisci are slightly concave to accommodate the condyles of the femur and providing greater contact surface area. The medial meniscus is larger than the lateral and more ovoid in shape. Anteriorly, it is thin and pointed at its attachment in the anterior intercondylar area of the tibia, directly outside the anterior cruciate ligament. Posteriorly, it is broadest, attaching in the corresponding posterior fossa, anteriorly to the origin of the posterior cruciate ligament. The lateral meniscus is smaller and more circular, its anterior horn being attached in the anterior intercondylar area, posteriorly and laterally to the insertion of the anterior cruciate ligament. Its posterior horn terminates in the posterior intercondylar area, immediately anterior to the termination of the posterior horn of the medial meniscus. The lateral meniscus is weakly attached around the margin of the lateral tibial condyle, except where crossed by the popliteal tendon and is not attached to the fibular collateral ligament. Near its posterior attachment, the lateral meniscus frequently sends off a collection of fibres which either join or lie behind the posterior cruciate ligament. The bundle of fibres, termed the posterior meniscofemoral ligament, terminates in the medial condyle of the femur immediately behind the attachment of the posterior cruciate ligament. Depending upon whether it passes anteriorly or posteriorly to the posterior cruciate ligament, the ligament is known, respectively, as the ligament of Humphry or the ligament of Wrisberg. Occasionally, both meniscofemoral ligaments are present, their function apparently being to provide a secondary restraint to posterior tibial translation. Occasionally, an anterior meniscofemoral ligament is also present, with a similar but anterior relationship to the posterior cruciate ligament. The lateral meniscus is thus loosely attached to the tibia and has frequent attachment to the femur. Therefore, it tends to move forward and backward with the lateral femoral condyle during flexion of the knee. In contrast, the medial meniscus is more firmly fixed to the tibia. The convex anterior margin of the lateral meniscus is connected to the anterior horn of the medial meniscus (or its convex anterior margin) by the transverse genicular ligament. This connection allows the two menisci to move in unison. This ligament, which varies considerably in thickness, is often absent. The curved external margins of the menisci are attached to the fibrous capsule of the knee joint (and thus the synovial membrane) and through it, to the edges of the articular surfaces of the tibia. The capsular fibres attaching the meniscal margins to the tibial condyles are termed coronary ligaments. The medial meniscus is further restrained by its attachment to the deep surface of the tibial collateral ligament. The capsular and tibial attachments of the meniscus may be seen clearly in FIG. 9. The tibial plateaux and meniscal horn attachment sites are illustrated in FIG. 10.

With reference to FIG. 9, it can be seen that the native meniscus 1 is attached to synovial capsule 2 via ligamentary connection 3 and thence, via said synovial capsule, to tibial articular cartilage 4. Femoral articular cartilage 5 has freedom of movement in relation to said meniscus and said tibial articular cartilage. It will be seen that a space exists between the meniscus proper and the synovial membrane, this space, normally occupied by ligamentary connection 3, becoming available when said native meniscus and said ligamentary connection are removed.

The location and morphology of the menisci and associated structures are illustrated in FIG. 3.

The anterior glide of the femoral condyles during flexion is also influenced by the menisci. The effective ‘wedging’ effect of the menisci acts to restrain the condyles from posterior displacement while the reaction forces applied to them act to displace the menisci posteriorly on the tibial plateaus. Deformation of the menisci occurs because the rigid attachment of their horns limits their ability to move in their entirety. Posterior deformation permits the menisci to remain beneath the femoral condyles as the condyles move on the tibial plateaus. As the knee returns to extension from full flexion, the menisci return to their neutral positions and, as extension continues, are deformed anteriorly. Appropriate posterior deformation of the menisci is assisted by muscular mechanisms. During knee flexion, for example, through its attachment to posterior horn of the medial meniscus, the semimembranous applies a force to the medial meniscus urging it posteriorly. An investigation has found that, in more than 40 percent of knees, the semimembranous has a similar attachment to the posterior horn of the lateral meniscus. The popliteus applies a similar force to the lateral meniscus.

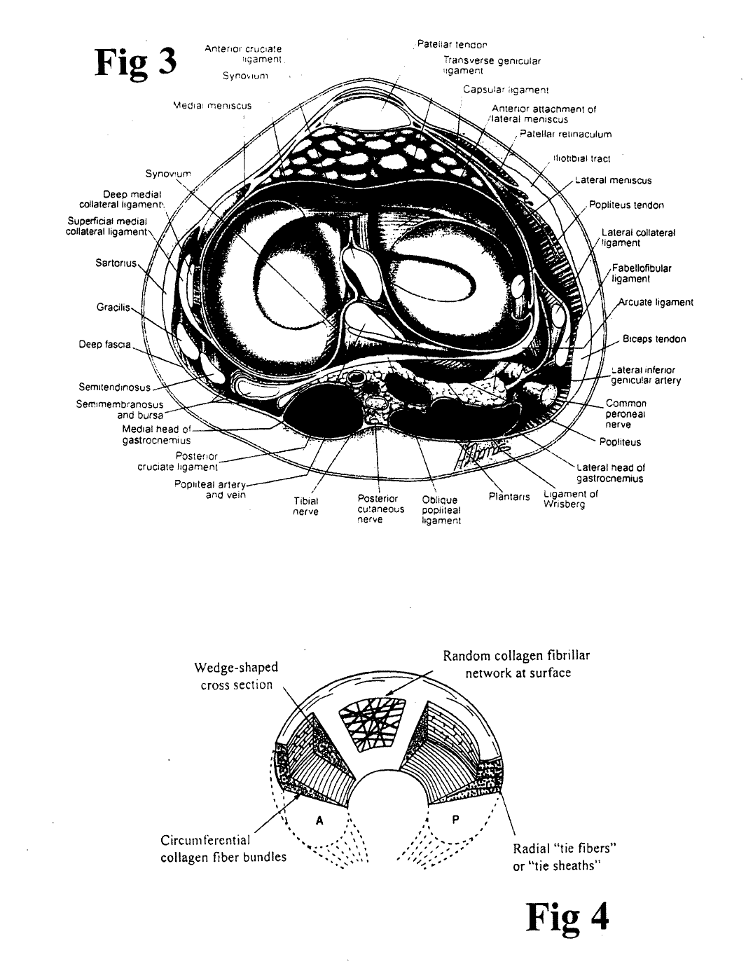

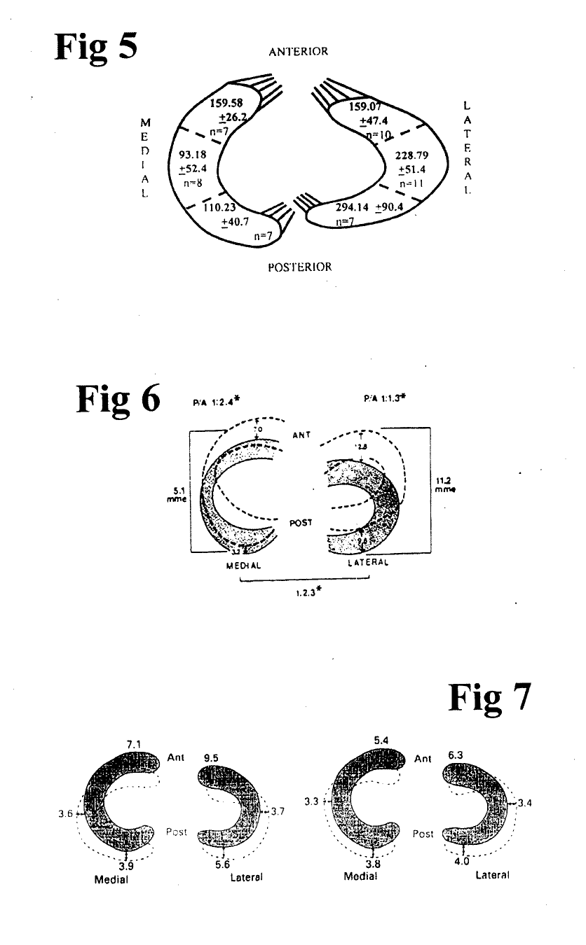

With reference to FIG. 4, the physical structure of the meniscal collagen networks can be roughly divided into three separate zones. In the outer, superficial layer, fibrils are randomly oriented and are interwoven to form a fine mesh. Immediately beneath this mesh is a narrow zone in which the collagen bundles show a much more irregular orientation. Interior to these two surface zones, the collagen fibres form large bundles that can be seen with the naked eye. These fibre bundles are circumferentially arranged, extending from the anterior attachment site to the posterior attachment site. Between these large, circumferentially arranged collagen fibre bundles are smaller tie fibres or tie sheaths orientated radially and extending from the periphery to the inner edge. Thus, compressive force applied to the meniscus is translated into a circumferentially directed tensile or hoop dress, supported by the strong circumferential fibres that dominate its ultrastructure. With reference to FIG. 5, Young's modulus (or tensile modulus) values for locations within the human menisci in tension are shown (values in MPa). It will be noted that the values are well below those of most polymer materials. For example, Kevlar® (aramid) has a tensile modulus normally in the range 83 to 186 GPa. Viscoelastic behaviour of meniscus material to tensile and compressive forces is complex, the tensile modulus, stiffness and failure stress correlating with collagen content and ratio of collagen to proteoglycan (PG). When meniscus material is loaded in compression, a loss of volume can occur due to fluid exudation from the tissue and/or fluid distribution within the tissue. The concentration of PG within the tissue has been shown to affect permeability, suggesting a direct relationship between PG content and compressive stiffness. The concentration and molecular conformation of proteoglycan aggregates in cartilage vary with age and disease and the amount of PG present depends on joint loading and motion. In general, with aging and disease, the size of the PG aggregates decreases by shortening of the hyaluronic acid chain or by shortening of either the protein core or glycosaminglycan chains, or both. Another important age-related change in PG can be observed at the molecular level. Chondroitin sulphate (CS) has two isomeric forms, CS₄ and CS₆, where the subscript indicates the location of sulphation on the hexosamine. It has been observed that the CS₄ isomer is more common in young cartilage, whereas the presence of CS₆ isomer increases with age. The net result is a reduction in resilience of the cartilage and a concomitant disposition towards mechanical damage.

Examination of the knee joint using magnetic resonance imaging has shown the relatively large excursions experienced by the menisci during various phases of knee joint flexion. FIGS. 6, 7 and 8 give some figures in this regard. Given the relatively high degree of meniscal mobility, it will be appreciated that the primary locational mechanism of the femoral condyles on the tibial plateaux is tension applied by the anterior and posterior cruciate ligaments. The menisci essentially provide a moveable, cushioned bearing surface for the femoral condyles and may, at extremes of knee flexion, provide supplementary locational assistance. This factor makes possible the provision of prosthetic menisci which, while not able to accommodate the rigors of athletic performance, will readily meet the needs of a sedentary person of middle age.

Meniscus failure commonly takes two forms: direct mechanical damage and that resulting from degenerative breakdown. In sports persons, for example, acute tearing of the meniscus may result when the knee is bent and forcefully twisted. Degenerative tears in the meniscus are very common in older persons with some 60 percent of western populations over the age of 65 years having some sort of degenerative breakdown. While acute tearing may result in the sudden onset of symptoms, in older subjects, degenerative breakdown may result from minor events and be symptomless for an extended period. A combination of circumstances, such as age-related degenerative changes, ‘wear and tear’ arthritis of the whole knee typically found in former athletes, inflammatory arthritis, decline of synovial lubrication, degradation caused by enzymes, unnatural gait, alignment disorders of the leg or excessive knee loadings as a result of occupational activities may result in progressive frictional wear of a meniscus, the cartilage having very little power of natural restoration. The meniscus is capable of self healing only in the vascularised, innervated peripheral zone while the unattached central zone is nourished only by synovial fluid and, generally speaking, is incapable of self healing.

In light of the foregoing generally, it is postulated that, if arthritic deterioration can be detected at an early enough stage, intervention in the form of the implantation of prosthetic menisci may be sufficient to almost immediately restore normal knee function. Further, this has the potential to arrest the deterioration process and to forestall the eventual need for total or partial knee replacement. If the implantation procedure can be performed arthroscopically as a day procedure, the reduction in demand for hospital bed space and orthopaedic, anaesthetic, physiotherapy and general medical services will represent a significant cost saving. Concomitant benefits would be rapid patient recovery with minimal discomfort and, if required, ease of revision.

Primary objects of the present invention are to provide prosthetic menisci to replace the natural human knee menisci, together with surgical procedures for removal of the natural menisci and implantation of the prostheses; the prostheses being readily matchable to the dimensions of the femoral condyles, able to be securely located on the tibial plateaus and to replicate normal meniscal motion while providing durable working surfaces of low friction; having compatibility with the constituents of synovial fluid, and being capable of accommodating the stresses imposed by the knee working under all normal loads. Secondary objects of the present invention are the provision of a prosthetic meniscus which may be implanted using arthroscopic surgical procedures, the surgical procedures and effect of the prosthetic menisci being such as to require minimal rehabilitation for a patient.

According to the present invention, prosthetic menisci of correct planform shaping are made in hollow form from suitable materials, reinforced and treated chemically and/or physically to render their surfaces attractive to the lubricating constituents of synovial fluid. Access is gained to the knee compartment via minimal incisions and displacement of the tendinous and capsular tissue surrounding the joint. The natural menisci are removed by surgically severing all of their tibial and capsular attachments with careful attention to haemostasis. A prosthetic meniscus is implanted by collapsing it into compact form, introducing it into the appropriate joint compartment, correctly positioning it between the femoral condyle and tibial plateau and securing it in place by suitable means. While the femoral condyle and tibial plateau are maintained in their correct relationship, a settable resin catalysing to a gel with a rubbery consistency is injected into said prosthetic meniscus, expanding said meniscus such that it shapes itself to the adjacent bony surfaces. The femoral condyle and tibial plateau are maintained in their correct relationship until said settable resin has substantially catalysed. The synovial capsule is then modified as required to fully enclose the joint, separated tissue is reinstated and the skin incisions are closed.

The various aspects of the present invention will be more readily understood by reference to the following description of preferred embodiments given in relation to the accompanying drawings in which:

FIGS. 1(a) and 1(b) are lateral, diagrammatic views of the bones of the knee during extension and flexion;

FIG. 2 is a superior, diagrammatic view of the proximal end of the tibia of the right knee;

FIG. 3 is a superior, diagrammatic, transverse cross-sectional view through the right knee just above the menisci;

FIG. 4 is a fragmentary, diagrammatic view of a meniscus partially sectioned to display its internal structure;

FIG. 5 is a diagrammatic presentation of regional variations of Young's Modulus in the human meniscus in tension;

FIG. 6 is a superior, diagrammatic view of displacement of menisci on the tibial plateaux at 0 and 120 degrees of knee flexion, the displaced positions depicted in solid line¹;

FIG. 7 is a superior, diagrammatic view of displacement of menisci on the tibial plateaux during 90 degrees of flexion from an erect stance with weight bearing and during 90 degrees of flexion in a sitting position, relaxed and bearing no weight, the displaced positions depicted in broken line²;

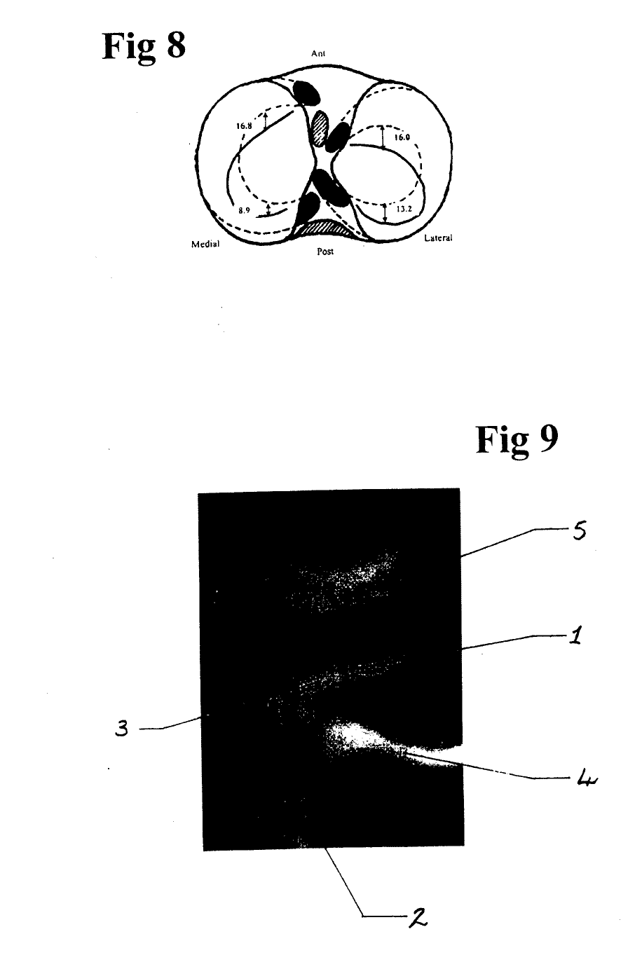

FIG. 8 is a superior, diagrammatic view of displacement of the meniscal inner margins on the tibial plateaux during deep knee flexion, the displaced margins depicted in solid line³;

FIG. 9 is a fragmentary cross-sectional view on a sagittal plane of the femoral condyle, meniscus and tibial plateau, separated to allow an appreciation of their relative dimensions;

FIG. 10 is a superior, diagrammatic view of the proximal end of the tibia of the right knee;

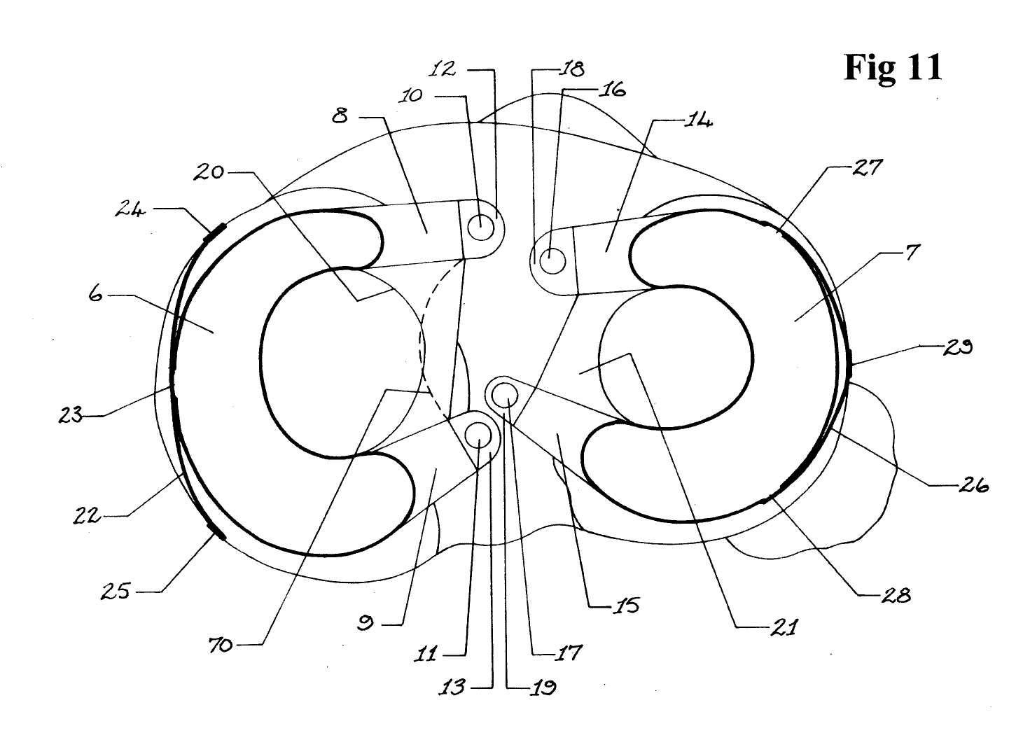

FIG. 11 is a superior, diagrammatic view of the proximal end of the tibia of the right knee depicting prosthetic menisci anchored via horns and supplementary locating means;

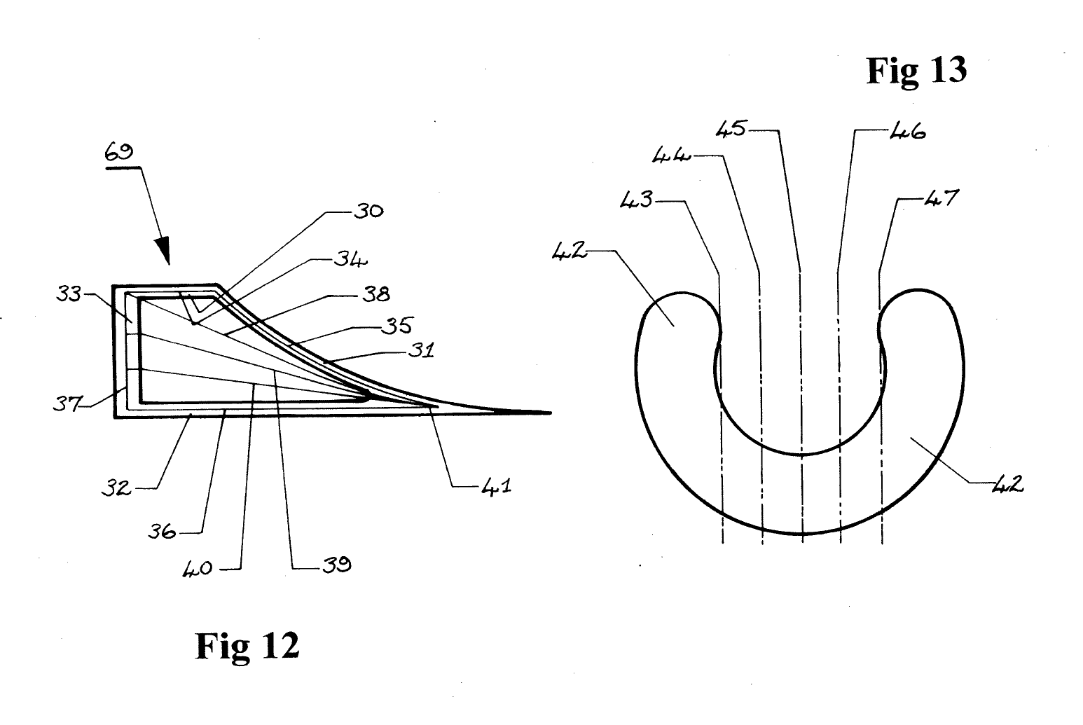

FIG. 12 is a diagrammatic, transverse cross-sectional view of a hollow prosthetic meniscus to be expanded by filling with a suitable settable resin;

FIG. 13 is a diagrammatic plan view of the meniscus of FIG. 12 showing folding lines;

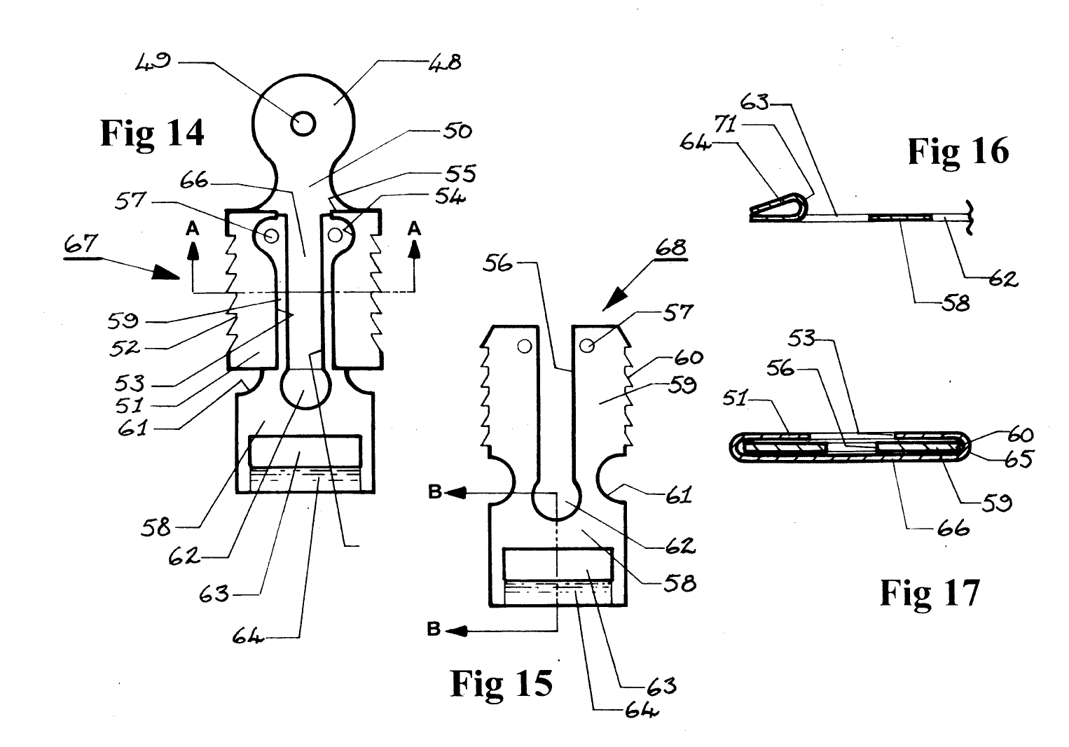

FIG. 14 is a plan view of a coupling assembly to connect a horn of a prosthetic meniscus to the tibia;

FIG. 15 is a plan view of the male part of the coupling assembly of FIG. 14;

FIG. 16 is a fragmentary, longitudinal cross-sectional view on B-B of the male part of the coupling assembly of FIG. 15;

FIG. 17 is a transverse cross-sectional view on A-A of the coupling assembly of FIG. 14.

The figures are drawn to a variety of scales and no meaning or significance should be adduced from this fact.

With reference to FIGS. 3 and 10, the positioning and attachment of the horns of the lateral and medial menisci are depicted. With further reference to FIGS. 6¹ and 7² and particularly to FIG. 8³, it may be seen that, with progressive flexion of the knee joint, the menisci are progressively translated posteriorly. This is accompanied, to varying degrees, by posterolateral displacement in the case of the lateral meniscus and posteromedial displacement in the case of the medial meniscus. As the menisci are securely anchored by their horns, the result of such displacement is an elastic distortion of the menisci from their natural shapes and a stretching of the horns with a concomitant reduction in their heights. An inspection of FIGS. 1 and 2 will give a better understanding of factors affecting meniscal displacement or translation. The elastic distortion of the menisci appears to be such as to permit a very rapid recovery of the menisci to their relaxed positions during rapid and repeated flexion of the knee joint. It can be argued that this facility is a natural development almost wholly applicable to the young and physically active, and that a person of middle-age or older following a sedentary lifestyle has little need of it. For older persons, providing the menisci are constrained within a suitable range of translation with their positioning regulated by condylar movement, they will adequately perform their principal functions of providing shock absorption and an enlarged bearing surface area for the joint. Limitation of meniscal translation to a range corresponding to a maximum knee joint flexion of 120° will accommodate most sedentary activities. The principal means employed to effect such translational constraint of the menisci, in addition to anchoring of the horns, are the supplementary locating straps described in relation to FIG. 11.

With reference to FIG. 11, prosthetic menisci 6, 7 are made hollow as described in relation to FIG. 12 and are free to move about on the tibial plateaus anchored, respectively, by anterior and posterior horns 8, 9 and 14, 15. In the preferred embodiment, the ends of said horns incorporate fixing plates 12, 13, 18, 19 made from a suitable metal alloy material. Said fixing plates are located in suitable recesses (not shown) cut into the proximal tibial surface and each is secured in place by one or more suitable fastenings 10, 11, 16, 17 passing through said plates into the bone. In the preferred embodiment, said horns are made from a suitable biocompatible elastomer and are strengthened by reinforcements (not shown) in the form of monofilaments or spun or braided, multi-filament yarns made from flexible materials having a suitable tensile strength. In the preferred embodiment, said reinforcements are made from Kevlar® and have a thickness in the range 0.01 to 1.0 millimetres. Said reinforcements are embedded in said horns in one or more layers and, to permit elastic extension of said horns in the range 10 to 50 percent, adopt an approximately sinusoidal form in their relaxed state. In the preferred embodiment, said sinusoidal form is characterised by a wavelength and amplitude in the range 1.0 to 6.0 millimetres. In the preferred embodiment, said reinforcements pass into said prosthetic menisci to form a strong connection between the two and pass through suitable apertures (not shown) in said fixing plates and are doubled back on themselves to form a strong connection between said horns and said plates, said reinforcements being fully encapsulated in the elastomeric material of said horns. In the preferred embodiment, said apertures in said fixing plates are made with rounded edges to prevent chafing of said reinforcements. Webs 20, 21 are optionally provided to join, respectively, the horns of prosthetic menisci 6 and 7 to provide a degree of control of the shape of said prosthetic menisci. The inner edges of said webs are preferably shaped such that, together with the inner edges of said menisci, they create roughly circular apertures. Said webs are optionally made porous or foraminous, suitably reinforced and having a maximum elastic extension ranging from zero to 20 percent. In the preferred embodiment, the upper and lower surfaces of said prosthetic menisci, said horns and said webs are treated as described herein to improve their lubrication by synovial fluid. In an alternative embodiment (not shown), said webs are deleted and the interior area of each said prosthetic meniscus is filled with an apron of thin, porous sheet material, the typical inner edge of which is depicted in broken line as 70. The edges of said apron are strongly attached to said menisci and to said horns, said apron taking the form of film or woven sheet made from a strong, cross-linked polymer material with a thickness in the range 0.05 to 0.5 millimetres and permitting elastic extension in the range zero to 20 percent. Where said apron material is not woven and therefore not porous, a large plurality of small apertures is provided in it to permit a free flow of synovial fluid said material. In the preferred embodiment, said apertures are round or approximately round with a diameter in the range 0.25 to 3.0 millimetres with a spacing one to another in the range 0.25 to 5.0 millimetres. Also in the preferred embodiment, said apertures are arranged in patterns which create a plurality of uninterrupted stress transmission paths passing fully across the widths of said prosthetic menisci, each said stress transmission path having an angular separation from adjacent paths in the range 15° to 45°. Also in the preferred embodiment, said apron material is treated to reduce friction between itself and abutting biological surfaces or to improve its lubrication by synovial fluid, both as detailed elsewhere herein.

Said prosthetic menisci are free to move about on the tibial plateaus anchored, respectively, by anterior and posterior horns 8, 9, 14, 15 and located by supplementary locating straps 22, 26. Said locating straps are made with limited elastic extensibility in the range 10 to 50 percent and of a reinforced material of similar nature to that of said horns. Said locating straps are optionally orientated in two ways. As depicted in the figure, locating strap 22 is fixed to the outer edge of medial prosthetic meniscus 6 at a single, more or less centrally (medially) located point 23, its ends being fixed to anchors 24, 25 which are, in turn, fixed to and project above the edges of the tibial plateau in antero-medial and postero-medial positions. If necessary, bone is removed from the edges of said tibial plateau to provide suitable attachment faces for said anchors. Also as depicted in the figure, the ends of locating strap 26 are fixed to the outer edge of lateral prosthetic meniscus 7 in antero-lateral and postero-lateral positions 27, 28, the central part of said locating strap being fixed to anchor 29 which is, in turn, fixed to and projects above the edges of the tibial plateau in a medial position. If necessary, bone is removed from the edge of said tibial plateau to provide a suitable attachment face for said anchor. In the preferred embodiment, said reinforcement material of said locating straps passes through suitable apertures in said anchors and is doubled back on itself to form a strong connection between said locating straps and said anchors, said reinforcement material being fully encapsulated in the elastomeric material of said locating straps. In the preferred embodiment, the edges of said anchor apertures are made with rounded edges to prevent chafing of said reinforcement material.

With reference to FIG. 12, a prosthetic meniscus 69 of the present invention is made hollow and comprises condylar contact panel 31, upper panel 30, outer panel 33 and tibial contact panel 32. Sheets of reinforcement material 34, 35, 36, 37 are embedded in said panels to provide additional tensile strength. One or more internal panels (three depicted) 38, 39, 40 of similar reinforcement material are optionally fixed at their outer edges to outer panel reinforcement sheet 37 and converge to line 41 where they join and are embedded. In its normally loaded, functional form, said internal panels of reinforcement material act to minimise extrusion of said prosthetic meniscus from a joint. Said reinforcement material takes the form of a thin, flexible sheet material, such as Kevlar®. Said material ranges in thickness from 0.005 to 0.1 millimetre and its thickness and extent are optionally varied according to the location within said prosthetic meniscus. In the preferred embodiment, a plurality of apertures (not shown) is provided in said sheet material, in that of panels 30, 31, 32, 33 to enhance its engagement with the material of said panels, and in that in the hollow interior of said prosthetic meniscus, to facilitate the distribution of a settable resin injected into said interior. Said apertures are of any suitable size and shape and of an arrangement such as to leave intact zones capable of satisfactorily carrying the radial and circumferential loads applied to said sheet material. In the preferred embodiment, the parts from which said hollow meniscus is fabricated are bonded together using one of the permanent biocompatible adhesives which are well known in the art. In the preferred embodiment, one or more moulds are created (or selected from an available range of moulds) for the required size and final shaping of a specific prosthetic meniscus.

With additional reference to FIG. 13, in its implantation via an arthroscopic procedure, orientated as required and extruded into the knee joint. Varus or vagus force is applied as necessary to open the joint. Within the knee joint, said prosthetic meniscus is unfolded and positioned correctly, its horns being extended and their fixing plates (depicted as 12, 13, 18, 19 in FIG. 11) secured to the tibia in the manner described in relation to FIG. 11. With the femoral condyles positioned correctly in relation to the tibia, said prosthetic meniscus is inflated to the shape of the native meniscus by injection of a suitable liquid resin through outer panel 70 by means of a suitable syringe, said liquid being pre-mixed with a catalyst which causes it to rapidly set into a rubbery gel of suitable elasticity and hardness. Said liquid is optionally injected at multiple sites between panels of reinforcement material 38, 39, 40 or at a single site with distribution and equalisation taking place via said apertures in panels of reinforcement material 38, 39, 40. The femoral condyles are maintained positioned correctly in relation to the tibia until gelation of said liquid is substantially advanced. Access is gained to the knee compartment via minimal incisions and separation or displacement of the tendinous and capsular tissue surrounding the joint. The natural menisci are removed as required by surgically severing all of their tibial, ligamentary and capsular attachments with careful attention to haemostasis, a process well known in the art. Where only one said natural meniscus is removed, the transverse geniculate ligament is severed at an appropriate length and sutured to the base of the anterior cruciate ligament. Said prosthetic menisci are selected for planform size and shape from radiographic, ultra-sonic or magnetic resonance-derived images of the condyles, although some success has been demonstrated in the selection of allograft meniscal replacements simply in relation to such factors as sex and height of a subject. In an alternative embodiment, a standard prosthetic meniscus of suitable size is given final shaping by laser ablation. At completion of the procedure, the synovial capsule is modified as required to fully enclose the joint, separated tissue is reinstated and the skin incisions are closed.

With reference to FIGS. 14, 15, 16, 17, fixing plate 48 is formed on the distal end of neck part 50 which is, itself, an extension of lower panel 66 of coupling assembly female part 67. Aperture 49 is provided in said fixing plate to accommodate a suitable fastening. Said female part is formed by the side parts 51 of said lower panel being folded through 180° to create a narrow space between the adjacent surfaces of said folded side parts and said lower panel. Longitudinal gap 53 is formed between the parallel adjacent edges of said folded-in side parts, the ends 55 of said folded side parts being turned down to provide stops at the distal end of said narrow space. A plurality of suitably spaced cuts is made in the side edges 65 of said female part normal to its longitudinal axis and material adjacent said cuts is pressed inwardly to create internal sprags 52. Coupling assembly male part 68 comprises base 58 upon which are formed coupling bars 59. Said coupling bars are separated by parallel slot 56 and their elastic movement in relation to base 58 is facilitated by removal of material at side recesses 61 and recess 62 at the base of slot 56. A plurality of ratchet-type teeth 60 is provided along the edges of said coupling bars, said teeth being orientated to engage sprags 52 when the two said coupling assembly parts are engaged. Apertures 57 are provided at the distal ends of said coupling bars, a suitable plier-type tool being engaged with said apertures as required to deflect said coupling bars inwardly such that said ratchet-type teeth disengage from said sprags, thereby allowing said coupling assembly male part to be withdrawn from said coupling assembly female part. Slot 53 provides access for said tool during said withdrawal. Recesses 54 are formed in the distal side edges of slot 53 to facilitate access to apertures 57 by said tool. It will be appreciated that said coupling assembly can be made in a variety of lengths, widths, thicknesses, aspect ratios and materials to suit a variety of applications. Transverse slot 63 is created in base 58 by cutting and folding back a strip of material 64 to form rounded edge 71. The attachment of the horn of a prosthetic meniscus passes through said slot and is doubled back over said rounded edge, said rounded edge acting to minimise chafing effects.

Said prosthetic menisci are moulded from a suitable biocompatible elastomer (the base material), in the preferred embodiment, the elastomer being DSM-PTG Carbosil® 20 90A biocompatible silicone polycarbonate urethane, manufactured by DSM Biomedical, of 6167 RA Geleen, The Netherlands. The principal mechanical properties of the material are:

| Density | 1.16 | g/cc |
|---|---|---|
| Hardness, Shore A | 90 | |
| Tensile Strength, Ultimate | 42.6 | MPa |
| Tensile Strength, Yield | 6.4 | MPa |
| Elongation at Break | 530% | |
| Flexural Modulus | 0.0407 | GPa |
| Flexural Strength, yield | 1.90 | MPa |
| Tear Strength | 87.7 | kN/m |

| | |
|---|---|
| Taber Abrasion, mg/1000 Cycles | 57.0 |
| Compression Set | 15.0% |

The material combines the biocompatibility and biostability of conventional silicone elastomers with the processability and toughness of thermoplastic urethane elastomers. The material is non-cytotoxic and non-haemolytic, has a low-energy silicone surface, has outstanding oxidative stability, is hydrophobic, has high tensile strength and is optically clear.

PurSil™ silicone-polyetherurethane and CarboSil™ silicone-polycarbonateurethane are true thermoplastic copolymers containing silicone in the soft segment. These high-strength thermoplastic elastomers are prepared through a multi-step bulk synthesis where polydimethylsiloxane (PSX) is incorporated into the polymer soft segment with polytetramethyleneoxide (PTMO) (PurSil) or an aliphatic, hydroxyl-terminated polycarbonate (CarboSil). The hard segment consists of an aromatic diisocyanate, MDI, with a low molecular weight glycol chain extender. The copolymer chains are then terminated with silicone (or other) Surface-Modifying End Groups™. Aliphatic (AL) versions of these materials, with a hard segment synthesized from an aliphatic diisocyanate, are also available. PurSil and CarboSil can be melt fabricated by conventional extrusion, injection molding, or compression molding techniques. Rod, pellet, and tubing extruded from these materials displays an excellent surface finish and low gel content. In addition, these materials are heat-sealable, readily blended with fillers, and easily post-formed. In an alternative embodiment, said elastomer is Tecoflex® SG-93A thermoplastic polyurethane elastomer (polyether), manufactured by Lubrizol Advanced Materials, Inc., of Cleveland, Ohio, USA, which has a nominal Shore A hardness of 87. This material is formulated especially for solution moulding. In other alternative embodiments, elastomer materials similar in characteristics to the Carbosil and Tecoflex products and having a hardness in the Shore A range 60 to 95 are used with the present invention.

In alternative embodiments, said prosthetic menisci are made from one or more of the synthetic polypeptide materials of the type taught by Keeley et at in Patent No. WO 2008/140703 A2⁵. These materials comprise at least three consecutive beta-sheet/beta-turn structures and at least one crosslinking amino acid residue that participates in crosslinking, wherein the crosslinking residue is distinct from the beta-sheet/beta-turn structures, each polypeptide is between 150 and 500 amino acids in length and the material is a solid or liquid. In particular aspects, each beta-sheet structure may comprise from 3 to about 7 amino acid residues. In some embodiments, the amino acid sequences of the crosslinked polypeptides are the same; while in other embodiments the amino acid sequences of the crosslinked polypeptides are different. In some embodiments, the material further comprises a reinforcing material, such as animal material, a synthetic material or metal. In other embodiments, the material further comprises a non-protein hydrophilic polymer. In some embodiments, the material further comprises glycosaminoglycan moieties, such as hyaluronan moieties. In some embodiments, the material comprises a mixture of crosslinked polypeptides and glycosaminoglycan moieties. In other embodiments, the crosslinked polypeptides are covalently linked to the glycosaminoglycan moieties. In some embodiments, the material is solid and may be in the form of pads, sheets and ligament-like structures. In other embodiments, the material is a liquid, such as a solution or suspension.

In alternative embodiments, said prosthetic menisci are made from hydrophilic polymer materials which exhibit a high degree of biocompatibility. In the preferred embodiment, said materials are hydrogels in which water absorption has been reduced and firmness increased by reduction of the proportion of hydrophilic monomers, by incorporation of hydrophobic comonomers or by an increase in the degree of crosslinking. Said prosthetic menisci are optionally made monolithic or with coatings of a similar material having different characteristics, such as greater hardness. Suitable hydrogels are those based upon methacrylate and acrylate, such as polyhydroxyethylmethacrylate; those based upon polyvinyl alcohol; those based upon polyethylene glycol, including combinations with collagen, methylated collagen or a protein such as albumin cross-linked with a collagen compound and copolymers with condensation polymers such as Nylon 6 and polyurethane (Biopol); those based upon polyethylene oxide; and those based upon acrylamide or polyacrylamide, such as polyvinylpyrrolidinone or hydrolysed polyacrylonitrile (Hypan series of hydrogels). In other alternative embodiments, modified forms of natural hydrophilic polymers, such as collagen, alginate and carrageen are employed. Other such materials are those created by derivatizing cellulose, a polysaccharide containing reactive hydroxyl groups which can easily be substituted to form ethers. Typical of these materials are sodium carboxymethyl-cellulose and hydroxyethyl and hydroxypropyl-cellulose. The properties of these polymers depend upon the molecular weight and degree of substitution. Other compounds of which copolymers and combinations are optionally employed are types I, II and IV collagen; gelatin; agarose; cell-contracted collagen containing proteoglycans, glycosaminoglycans or glycoproteins; fibronectin; laminin; bioactive peptide growth factors; cytokines; elastin; fibrin; synthetic polymeric fibres made from poly-acids such as polylactic, polyglycolic or polyamino acids, polycaprolactones or polyamino acids. Any of the preceding compounds from which said prosthetic menisci are formed are optionally reinforced with collagen microfibrils created by the various methods known in the art, including electrospinning, or by lamination with one or more thin layers of high-tensile material, as described elsewhere herein.

Said moulds for the final shaping of said prosthetic menisci are made with polished surfaces to provide a glass-smooth finish to said menisci upper and lower bearing surfaces. To improve lubrication of said menisci by synovial fluid, said bearing surfaces are treated using a method⁴ which renders them attractive to dipalmitoylphosphatidylcholine (DPPC) by impregnating said surfaces with poly[2-methacryloyloxyethyl phosphorylcholine-co-n-butylmethacrylate] [poly(MPC-co-BMA)]. [poly (MPC-co-BMA)] is a biocompatible, lipid-attracting polymer soluble in solvent systems which also dissolve many polyurethanes. DPPC is the most abundant phospholipid in synovial fluid. In said method, the polyurethane elastomer is immersed in an ethanol solution containing BMA (0.3 mol l⁻¹) and benzoic peroxide (1 wt % to BMA) as a polymerization initiator for 15 hours, resulting in a slightly swollen surface. The material is lightly washed with ethanol and then immersed in an ethanol solution containing MPC (0.3 mol l⁻¹) for 30 minutes. After removal from the second solution, the material is blotted dry and then heated at 70° C. for 5 hours under an argon atmosphere to polymerize the monomers present in the surface of the material. Finally, the material is washed with ethanol and then dried en vacuo at room temperature for 24 hours. The method is adapted as required for volume application.

In an alternative embodiment (not shown), the smooth polymer working surfaces of said prosthetic menisci are coated directly with a layer of diamond-like carbon (DLC) of suitable thickness. DLC is harder than most ceramics, is bioinert and has a low frictional coefficient. Where necessary, said surfaces are modified prior to deposition by ion-implantation of nitrogen, O₂ plasma treatment, or the like. In the preferred embodiment, the coating is made using pulsed laser deposition, the radio-frequency plasma CVD process or ion beam-assisted deposition, the last-named being the most preferred. Where necessary, a smooth, thin, flexible layer of a harder polymer material is bonded to said working surfaces to provide a better substrate for said coatings. In the preferred embodiment, said layer of harder material has a thickness in the range 0.05 to 0.5 millimetre, the material being a cross-linked polymer of high tensile strength, such as Kevlar®. CVD coatings have been successfully demonstrated on a wide variety of polymer materials and the above-named methods can be performed at acceptable temperatures. In an alternative embodiment (not shown), said polymer surface layers to be coated are densely impregnated with carbon nanofibres with which said DLC coating makes a strong bond.

In an alternative embodiment (not shown), the smooth polymer working surfaces of said prosthetic menisci are coated with a thin layer of a wear-resistant material in the form of a suitable carbide, nitride or oxide. In the preferred embodiment, said layer is deposited from a suspended solution of nanoparticles using the electroless plating process. In a first embodiment, said surfaces are first plasma treated and a metal catalyst then infused into them by chemisorption to activate said surfaces. Typical metal catalysts are SnCl₂ and PdCl₂. In a second embodiment, said catalysts are infused into said surfaces using super-critical carbon dioxide (scCO₂), taking advantage of the solvency and plasticization effects of scCO₂. Where necessary, a smooth, thin, flexible layer of a harder polymer material is bonded to said working surfaces to provide a better substrate for said coatings. In the preferred embodiment, said layer of harder material has a thickness in the range 0.05 to 0.5 millimetre, the material being a cross-linked polymer of high tensile strength, such as Kevlar®.

In an alternative embodiment (not shown), the lubricity of said working surfaces of said prosthetic menisci is enhanced by the generation thereon of a layer of hyaline cartilage. In this embodiment, a suitable highly porous scaffold material is prepared and fused or otherwise fixed to said working surfaces. Suitable materials for said scaffold include synthetic hydrogels created by the graft polymerization of either hydroxyethyl methacrylate (HEMA) and methyl methacrylate (MMA) or HEMA and glycol dimethacrylate (GDMA) onto soluble collagen using different cross-linking agents; polyglycolic acid (PGA) extruded and assembled into fibrous form using textile processing techniques; poly(D,L-lactide-co-glycolide) (PLG) assembled by the electrospinning method; free radical polymerization of a combination of hydrolysed collagen, acrylic acid (AA) acrylamide (AAm) and distilled water and crosslinked using N,N′-methylene bisacrylamide (MBA); porous materials such as (poly)ethylene glycol-terephthalate-(poly)butylene-terephthalate (PEGT/μPBT) assembled by the controlled deposition of molten co-polymer fibres in three-dimensional form by computer-controlled syringe; and many others well known in the art. Said scaffold is seeded with articular chondrocytes and cultured in vitro under appropriate conditions. During generation of said cartilage, suitably shaped moulds are applied to the surface of said scaffold for periods to ensure generation of the desired surface shaping. Following generation of said cartilage, said prosthetic meniscus is implanted in the manner described herein.

In an alternative embodiment (not shown), said layers of cartilage are generated on the working surfaces of said prosthetic menisci using a method adapted from that taught by Kim et al in US 2010/0120149. In this embodiment, polymer scaffoldings are prepared and fused or otherwise fixed to said working surfaces and differentiated articular chondrocytes mixed with hydrogel are applied to said scaffoldings in liquid form and gelled. Said chondrocytes are then cultured in vitro under appropriate conditions. In this embodiment, said chondrocytes are first differentiated and clustered together to form cell aggregates of appropriate size using hanging drop culture, pellet culture, micromass culture, and rotational culture. Successful seeding of cell aggregates onto a polymer scaffold requires regulation of the average diameter of cell aggregates, typically in the range 10 to 800 μM. The number of cells used in formation of cell aggregates can be varied according to the type of cell used or the size of a single cell. For example, chondrocytes or bone marrow-derived mesenchymal stem cells are subjected to primary culture in the number of 1×10³ to 1×10⁷ cells, out of which 1×10³ to 1×10⁶ cells may be clustered together forming cell aggregates having an average diameter in the range 10 to 800 μM. Since the efficiency of chondrogenic differentiation is proportionate to the size of cell aggregates, it is necessary for cell aggregates to have an average diameter larger than a certain size, but not excessively larger than the pore size of the polymer scaffold, which would render it difficult to induce chondrogenic differentiation inside the polymer scaffold. The cell aggregates of differentiated chondrocytes are mixed with hydrogels in a solution state to form a cell aggregate-hydrogel complex in which the cell aggregates are evenly dispersed. The cell aggregates and hydrogels may be mixed in a weight ratio in the range of 1:1 to 1:100. This allows the establishment of a three-dimensional environment physiologically similar to that of natural cartilage. A smaller proportion of cell aggregates is unfavorable to cartilage regeneration, chondrogenesis-related ECM molecules secreted from the cell aggregates being unsuccessfully diffused and delivered, resulting in a slow progress in chondrogenesis. Suitable hydrogels for the present invention include, but are not limited to, fibrin, gelatin, collagen, hyaluronic acid, agarose, chitosan, polyphosphazine, polyacrylate, polyglactic acid, polyglycolic acid, pluronic acid, alginate, salts and the like, used alone or in mixture form. The complex of cell aggregates of differentiated chondrocytes evenly dispersed in the hydrogel matrix is applied in a solution state to a polymer scaffold and solidified into a gel state to obtain a cell aggregate-hydrogel-polymer scaffold complex. The gelation method depends upon the type of hydrogel used, the polymer scaffold providing a secure support for the hydrogel. The polymer scaffold is made from biodegradable and biocompatible polymers including, but not limited to collagen, gelatin, chitosan, alginate, hyaluronic acid, dextran, polylactic acid, polyglycolic acid, poly(lactic acid-co-glycolic acid), polycaprolactone, polyanhydride, polyorthoester, polyvinyl alcohol, polyethylene glycol, polyurethane, polyacrylic acid, poly-N-isopropylacrylamide, poly(ethyleneoxide)-poly(propyleneoxide)-poly (ethyleneoxide)copolymer, copolymers thereof and mixtures thereof. For the successful seeding of the cell aggregate-hydrogel complex onto the polymer scaffold, the polymer scaffold should have an interconnective porous structure with a uniform pore size typically in the range of 10 to 800 μM or, more specifically, 100 to 500 μM. Additionally, the polymer scaffold requires a porosity in the range 40% to 97%. If the porosity is not more than 40%, the pore interconnectivity is remarkably reduced, while if the porosity of the polymer scaffold exceeds 97%, the mechanical strength thereof is significantly lowered. Typically, the polymer scaffold should have a porosity in the range of 50% to 97% or, more specifically, 70% to 95%. The polymer scaffold can be prepared from the biocompatible polymers detailed above using methods well known in the art including, for example, casting/solvent extraction, gas foaming, phase separation, electrospinning, gel spinning, and the like. The cell aggregate-hydrogel-polymer scaffold complex created in the manner described has a structure in which the cell aggregates of differentiated chondrocytes are evenly dispersed in the hydrogel matrix, to form a cell aggregate-hydrogel complex, and the cell aggregate-hydrogel complex is immobilized onto the surface of the polymer scaffold while simultaneously filling up the pores thereof. The cell aggregate-hydrogel-polymer scaffold complex has the following advantages: it can efficiently induce chonrogenic differentiation due to the high degree of intracellular interaction resulting from the use of cell aggregates rather than single cells; the hydrogel creates a three-dimensional environment physiologically similar to that of natural cartilage; the cell aggregate-hydrogel-polymer scaffold complex further improves the efficiency of chondrogenic differentiation; the use of a polymer scaffold enables the maintenance of high mechanical strength accurate shaping, flexibility and uniform morphology during the chondrogenic differentiation; and the cell aggregate-hydrogel-polymer scaffold complex provides cartilage tissue with high mechanical strength, flexibility, and uniform morphology. In this embodiment, said polymer scaffold is first fused or otherwise fixed to said working surfaces of said prosthetic menisci before application of said cell aggregate-hydrogel complex in liquid form. In the preferred embodiment, said polymer scaffold typically has a thickness in the range 0.5 to 3 millimetres.

In another alternative embodiment (not shown), in which a scaffold material fused or otherwise fixed to said working surfaces of said prosthetic meniscus is seeded with articular chondrocytes, said working surfaces are prepared by the generation of a fluoridated hydroxyapatite coating on which is deposited a bone-like, microstructured beta-tricalcium phosphate layer. Said coating and said layer simulate the layer of calcified articular cartilage normally abutting subchondral bone. Said fluoridated hydroxyapatite coating is optionally infused into said working surfaces using super-critical carbon dioxide (scCO₂), taking advantage of the solvency and plasticization effects of scCO₂.

To improve the distribution of synovial fluid between the bearing surfaces of said prosthetic menisci and the femoral and tibial articular cartilage, a network of narrow channels is moulded into one or both said bearing surfaces. In the preferred embodiment, said channels have a width of between 0.25 and 2.0 millimetres, a depth of between 0.25 and 2.0 millimetres, have a part-spherical or other suitable cross-sectional shape, are separated by between 1.0 and 5.0 millimetres and are orientated more or less radially and circumferentially. Also for the same purpose, either or both said bearing surfaces are provided at some or all of the points of intersection of said channels with recesses orientated more or less normal to the surface at each point, having a depth of between 0.5 and 5.0 millimetres and a diameter of between 0.5 and 5.0 millimetres. For the same purpose, either or both said bearing surfaces are provided with recesses orientated more or less normal to the surface at each point, said recesses having a depth of between 0.5 and 5.0 millimetres, a diameter of between 0.5 and 5.0 millimetres and being separated from each other by a distance of between 0.5 and 10 millimetres.

In an alternative embodiment (not shown), either or both bearing surfaces of said menisci are provided with thin layers of a softer, more compliant base material, the thickness of said thin layers being preferably in the range 0.1 to 2.0 millimetres. By providing a more compliant bearing surface, this embodiment is better able to achieve microelasto-hydrodynamic lubrication.

Where magnetic resonance imaging is employed in the diagnosis of meniscal injury or deterioration or in the measurement of the size and shape of joint components, in the preferred embodiment imaging units employing knee coils of the several types manufactured by Fonar Corporation of 110 Marcus Drive, Melville, N.Y. 11747, USA are employed.

Any feasible combination of any part of the apparatus and/or any part of the method described herein should be taken to be disclosed by the specification.

### REFERENCES

1. Fu F. H., Thompson W. O. Motion of the Meniscus During Knee Flexion. In: Mow V. C., Arnoczky S. P., Jackson D. W., eds. Knee Meniscus: Basic and Clinical Foundations. New York: Raven Press, 1992: Chapter 6, FIGS. 20 and 21.
2. Vedi V., Williams A., Tennant S. J., Spouse E., Hunt D. M., Gedroyc W. M. W. (1998). Meniscal Movement: An In-vivo Study Using Dynamic MRI. The Journal of Bone & Joint Surgery (Br) 1999; 81-B37-41.
3. Hamamoto K., Tobimatsu M. D., Zabinska-Uroda K. (2004). Magnetic Resonance Imaging Evaluation of the Movement and Morphological Changes of the Menisci During Deep Knee Flexion. J. Phys. Ther. Sci. Vol. 16 No. 2, 2004
4. Williams III P. F., Powell G. L., Love B., Ishihara A., Nakabayashi N., Johnson R., LaBerge W. (1994). Fabrication and Characterization of Dipalmitoylphosphatidylcholine-attracting Elastomeric Material for Joint Replacements.
5. Keeley et al. International Patent No. WO 2008/140703 A2. Synthetic Peptide Materials for Joint Reconstruction, Repair and Cushioning.

1-61. (canceled)

62. Prosthetic knee menisci to be implanted in place of deteriorated native menisci to prevent damage to the articular cartilage of the femoral and tibial condyles and, thereby, to arrest the progressive development of osteoarthritis; said prosthetic menisci being made as hollow forms which are inflated after implantation by injection of a settable polymer to shape them into congruence with the femoral and tibial condyles; being sized for the femoral and tibial condylar surfaces; having internal reinforcement for strength and durability; being made from materials having elastomeric characteristics similar to those of native menisci; having bearing surfaces treated chemically and/or physically to improve the efficiency of lubrication by synovial fluid and to enhance the wear characteristics of the bearing surfaces; and being restricted in translation within the interarticular space by anchorage of their anterior and posterior horns and by the provision of secondary locating elements.

63. The prosthetic knee menisci of claim 62 in which the ends of the anterior and posterior horns incorporate fixing plates made from a suitable metal alloy material, said fixing plates being secured in place at the points of insertion of the native menisci by suitable fastenings passing through said fixing plates into the proximal tibial surfaces, recesses being created as required in the proximal tibial surfaces to accommodate said plates; optionally in which said secondary locating elements take the form of locating straps fixed to said menisci and to anchors fixed to the tibia; optionally in which said anterior and posterior horns and said locating straps are made from a suitable biocompatible elastomer strengthened by reinforcements in one or more layers which are fully encapsulated in the material of said horns and said locating straps and which permit elastic extension in the range 10 to 50 percent; optionally in which said reinforcements take the form of monofilaments or spun or braided, multi-filament yarns made from flexible materials such as aramid or para-aramid and having a suitable tensile strength and a thickness in the range 0.01 to 1.0 millimetres, said reinforcements adopting an approximately sinusoidal form in their relaxed state, said sinusoidal form being characterised by a wavelength and amplitude in the range 1.0 to 6.0 millimetres; optionally in which said reinforcements pass into and form a strong connection with said menisci.

64. The prosthetic knee menisci of claim 63 in which said reinforcements pass through suitable apertures in said fixing plates and said anchors, being doubled back on themselves to form a strong connection between said horns and said plates and between said locating straps and said anchors, said apertures in said fixing plates and said anchors being made with rounded edges to prevent chafing of said reinforcements.

65. The prosthetic knee menisci of claim 63 in which bone is optionally removed from the edges of the tibial plateau to provide attachment faces for the fixing of said anchors, said anchors projecting above the edges of the tibial plateau; optionally in which said locating straps are situated in the more or less annular zone normally occupied by the ligamentary connection joining the native meniscus to the synovial membrane; optionally in which said locating straps are fixed to said menisci at single, more or less centrally (medially) located points, their ends being fixed to said anchors which are, in turn, fixed to the edges of the tibial plateau in antero-medial and postero-medial positions; optionally in which said the ends of said locating straps are fixed to said menisci in antero-lateral and postero-lateral positions, the central points of said locating straps being fixed to single said anchors which are, in turn, fixed to the edges of the tibial plateau in a medial position.

66. The prosthetic knee menisci of claim 62 in which translation is limited to that corresponding to a maximum knee joint flexion of 120 degrees.

67. The prosthetic knee menisci of claim 62 in which the base material from which they are manufactured is a suitable biocompatible and biostable elastomer having a hardness in the Shore A range 60 to 95; optionally in which said base material is silicone-polyetherurethane or silicone-polycarbonateurethane, high-strength thermoplastic elastomers prepared by incorporating polydimethylsiloxane (PSX) into the polymer soft segment with polytetramethyleneoxide (PTMO) or an aliphatic, hydroxyl-terminated polycarbonate, the hard segment consisting of an aromatic diisocyanate, MDI, with a low molecular weight glycol chain extender, the copolymer chains being terminated with silicone or other Surface-Modifying End Groups; optionally in which said base material is thermoplastic polyurethane elastomer (polyether); optionally in which copolymer chains of said base material are terminated with silicone or other surface-modifying end groups.

68. The prosthetic knee menisci of claim 62 in which said menisci are made from synthetic polypeptide materials of the type taught by Keeley et al in Patent No. WO 2008/140703 A2, the materials comprising at least three consecutive beta-sheet/beta-turn structures and at least one crosslinking amino acid residue that participates in crosslinking, wherein the crosslinking residue is distinct from the beta-sheet/beta-turn structures and each polypeptide is between 150 and 500 amino acids in length; and/or in which each beta-sheet structure may comprise from 3 to about 7 amino acid residues; and/or in which the amino acid sequences of the crosslinked polypeptides are the same or different; and or in which the material further comprises a reinforcing material, such as animal material, a synthetic material or metal; and/or in which the material further comprises a non-protein hydrophilic polymer; and/or in which the material further comprises glycosaminoglycan moieties, such as hyaluronan moieties; and/or in which the material comprises a mixture of crosslinked polypeptides and glycosaminoglycan moieties; and/or in which the crosslinked polypeptides are covalently linked to the glycosaminoglycan moieties; and/or in which the material is solid and may be in the form of pads, sheets and ligament-like structures; optionally in which the base materials from which said menisci are made are hydrophilic polymer materials (hydrogels) in which water absorption has been reduced and firmness increased by reduction of the proportion of hydrophilic monomers, by incorporation of hydrophobic comonomers or by an increase in the degree of crosslinking, said prosthetic menisci being optionally made monolithic or with coatings of a similar material having different characteristics, such as greater hardness.

69. The prosthetic knee menisci of claim 62 in which the base materials from which said menisci are made are based upon methacrylate and acrylate, such as polyhydroxyethylmethacrylate; polyvinyl alcohol; polyethylene glycol, including combinations with collagen, methylated collagen or a protein such as albumin cross-linked with a collagen compound and copolymers with condensation polymers such as Nylon 6 and polyurethane (Biopol); polyethylene oxide; or acrylamide or polyacrylamide, such as polyvinylpyrrolidinone or hydrolysed polyacrylonitrile (Hypan series of hydrogels); optionally in which the base materials from which said menisci are made are modified forms of natural hydrophilic polymers, such as collagen, alginate and carrageen; or those created by the derivatizing of cellulose, such as sodium carboxymethyl-cellulose and hydroxyethyl and hydroxypropyl-cellulose; optionally in which the base materials from which said menisci are made are copolymers and combinations of types I, II and IV collagen; gelatin; agarose; cell-contracted collagen containing proteoglycans, glycosaminoglycans or glycoproteins; fibronectin; laminin; bioactive peptide growth factors; cytokines; elastin; fibrin; synthetic polymeric fibres made from poly-acids such as polylactic, polyglycolic or polyamino acids, polycaprolactones or polyamino acids; optionally in which the base material from which said menisci are made is optionally reinforced with collagen microfibrils created by the various methods known in the art, including electrospinning.

70. The prosthetic knee menisci of claim 62 in which final sizing and shaping is performed in a mould made or selected for the purpose, said mould being treated to provide a glass finish on the upper and lower bearing surfaces of said menisci.

71. The prosthetic knee menisci of claim 62 in which a first said internal reinforcement takes the form of sheet reinforcement material embedded in all walls of said hollow form to provide additional tensile strength; optionally in which a second said internal reinforcement takes the form of one or more internal panels of reinforcement material deployed within the interior of said hollow form, fixed at their outer edges to said embedded reinforcement material in the outer walls of said menisci and converging at the inner edges of said menisci where they join and are embedded, the second said reinforcement acting to minimise extrusion of said prosthetic meniscus from a joint; optionally in which said internal reinforcement takes the form of an aramid or para-aramid film in the thickness range 0.005 to 0.1 millimetre; optionally in which the thickness and extent of said reinforcement material varies according to the location within a said meniscus; optionally in which said reinforcement material is provided with a plurality of apertures, said apertures enhancing engagement of embedded said reinforcement material with the base material of said menisci, and facilitating the distribution of said settable resin injected into the interiors of said hollow form prosthetic menisci; optionally in which said apertures are of any suitable shape and of an arrangement such as to leave intact zones capable of satisfactorily carrying the radial and circumferential loads applied to said sheet material.

72. The prosthetic knee menisci of claim 62 in which, to better achieve microelastohydrodynamic lubrication, either or both said bearing surfaces of said menisci are provided with thin layers of a softer, more compliant base material, the thickness of said thin layers being in the range 0.1 to 2.0 millimetres; optionally in which the horns of a said prosthetic meniscus are optionally joined by webs to provide a degree of control of the shape of said prosthetic menisci, the inner edges of said webs being shaped such that, together with the inner edges of said menisci, they create roughly circular apertures; optionally in which said webs are optionally made porous or foraminous, suitably reinforced and having a maximum elastic extension ranging from zero to 20 percent, the upper and lower surfaces of said menisci, said horns and said webs being treated to improve their lubrication by synovial fluid; optionally in which said webs are flat braided from fine aramid or para-aramid fibres, their ends securely embedded in said meniscus horns, said webs preferably being encapsulated in said elastomer base material.

73. The prosthetic knee menisci of claim 62 in which the interior area of said prosthetic menisci is filled with an apron of thin, porous sheet material extending to the ends of said horns, the edges of said apron being strongly attached to said menisci and to said horns; optionally in which said apron takes the form of film or woven sheet made from a strong, cross-linked polymer material with a thickness in the range 0.05 to 0.5 millimetres and permitting elastic extension in the range zero to 20 percent; optionally in which, where the material of said apron is not porous, a large plurality of small apertures is provided in it to permit a free flow of synovial fluid through said material, said apertures preferably being round or approximately round with a diameter in the range 0.25 to 3.0 millimetres and with a spacing one to another in the range 0.25 to 5.0 millimetres; optionally in which said apertures are arranged in patterns which create a plurality of uninterrupted stress transmission paths passing fully across the widths of said prosthetic menisci, each said stress transmission path having an angular separation from adjacent paths in the range 15° to 45°; optionally in which said apron material is optionally encapsulated in said elastomer base material and treated to reduce friction between itself and abutting biological surfaces by improving its lubrication by synovial fluid.

74. The prosthetic knee menisci of claim 62 in which, to improve lubrication of said menisci by synovial fluid, said bearing surfaces are treated to render them attractive to dipalmitoylphosphatidylcholine (DPPC) by impregnating said surfaces with poly[2-methacryloyloxyethyl phosphorylcholine-co-w-butylmethacrylate] [poly(MPC-co-BMA)]; optionally in which a polyurethane elastomer is immersed in an ethanol solution containing BMA (0.3 mol¹) and benzoic peroxide (1 wt % to BMA) as a polymerization initiator for 15 hours to produce a slightly swollen surface, the material then being lightly washed with ethanol and immersed in an ethanol solution containing MPC (0.3 mol¹) for 30 minutes; after removal from the second solution, the material is blotted dry and then heated at 70° C. for 5 hours under an argon atmosphere to polymerize the monomers present in the surface of the material which is then washed with ethanol and then dried en vacuo at room temperature for 24 hours; optionally in which, to improve the distribution of synovial fluid between the bearing surfaces of said prosthetic menisci and the femoral and tibial articular cartilage, a network of narrow channels is moulded into one or both said bearing surfaces, said channels preferabl having a width of between 0.25 and 2.0 millimetres, having a depth of between 0.25 and 2.0 millimetres, having a part-spherical or other suitable cross-sectional shape, being separated by between 1.0 and 5.0 millimetres and being orientated more or less radially and circumferentially; optionally in which, to improve the distribution of synovial fluid, either or both said bearing surfaces are provided at some or all of the points of intersection of said channels with recesses orientated more or less normal to the surface at each point, said recesses having a depth of between 0.5 and 5.0 millimetres and a diameter of between 0.5 and 5.0 millimetres; optionally in which, to improve the distribution of synovial fluid, either or both said bearing surfaces are provided with recesses orientated more or less normal to the surface at each point, said recesses having a depth of between 0.5 and 5.0 millimetres and a diameter of between 0.5 and 5.0 millimetres, said recesses being separated from each other by a distance of between 0.5 and 10 millimetres.

75. The prosthetic knee menisci of claim 63 in which a said fixing plate is made in two separable parts, a female part fixed to the proximal tibial surface and a male part fixed to the horns of a said prosthetic meniscus to be received by and locked into said female part; optionally in which said female part is made with internally located sprags along each side which are engaged by complementary ratchet-type teeth formed along the edges of two parallel coupling bars of said male part, said coupling bars being able to be elastically displaced towards each other to disengage said teeth from said sprags; optionally in which said coupling bars are sprung together by a suitable plier-type tool engaging apertures in said coupling bars.

76. The prosthetic knee menisci of claim 62 in which the bearing surfaces of said prosthetic menisci are coated directl with a layer of diamond-like carbon (DLC) of suitable thickness, where necessary, said surfaces being modified prior to deposition by ion-implantation of nitrogen, O₂ plasma treatment, or the like, the coating being made by pulsed laser deposition, the radios-frequency plasma CVD process or ion beam-assisted deposition; optionally in which said bearing surfaces of said menisci are first densely impregnated with carbon nanofibres with which said DLC coating makes a strong bond; optionally in which the bearing surfaces of said prosthetic menisci are coated with a thin layer of a wear-resistant material in the form of a suitable carbide, nitride or oxide, said coating being deposited from a suspended solution of nanoparticles using the electroless plating process; optionally in which said surfaces are first plasma treated and a metal catalyst infused into them by chemisorption to activate said surfaces, said catalysts being SnCl₂ and PdCl; optionally in which said catalysts are infused into said surfaces using super-critical carbon dioxide (scCO₂), taking advantage of the solvency and plasticization effects of scCO₂; optionally in which, where necessary, a smooth, thin, flexible layer of a harder polymer material is bonded to said working surfaces to provide a better substrate for said coatings, said harder polymer material preferably having a thickness in the range 0.05 to 0.5 millimetre and taking the form of a cross-linked polymer of high tensile strength, such as ararnid or para-aramid.

77. The prosthetic knee menisci of claim 62 in which the lubricity of said bearing surfaces of said menisci is enhanced by the generation thereon of a layer of hyaline cartilage, said generation process comprising the preparation of a layer of suitable highly porous scaffold material and the fusing or otherwise fixing of said scaffold material to said bearing surfaces; the seeding of said scaffold material with articular chondrocytes; the culturing in vitro of said chondrocytes under appropriate conditions to generate said hyaline cartilage, suitably shaped moulds being applied to the surface of said scaffold for periods during said culturing process to ensure generation of the desired surface shaping; optionally in which said scaffold materials include synthetic hydrogels created by the graft polymerization of either hydroxyethyl methacrylate (HEMA) and methyl methacrylate (MMA) or HEMA and glycol dimethacrylate (GDMA) onto soluble collagen using different cross-linking agents; polyglycolic acid (PGA) extruded and assembled into fibrous form using textile processing techniques; poly(D,L-lactide-co-glycolide) (PLG) assembled by the electrospinning method; free radical polymerization of a combination of hydrolysed collagen, acrylic acid (AA) acrylamide (AAm) and distilled water and crosslinked using N,N′-methylene bisacrylamide (MBA); porous materials such as (poly)ethylene glycol-terephthalate-(poly)butylene-terephthalate (PEGT/uPBT) assembled by the controlled deposition of molten co-polymer fibres in three-dimensional form by computer-controlled syringe; and others well known in the art.

78. The prosthetic knee menisci of claim 62 in which the lubricity of said bearing surfaces of said menisci is enhanced by the generation thereon of a layer of hyaline cartilage, said generation process being adapted from that taught by Kim et al in US 2010/0120149 and comprising preparation of a layer of polymer scaffold material and the fusing or otherwise fixing of said scaffold material to said bearing surfaces; the application to said scaffold material in liquid form of differentiated articular chondrocytes mixed with hydrogel which is then gelled; and the culturing of said chondrocytes in vitro under appropriate conditions; optionally in which said polymer scaffold is made from biodegradable and biocompatible polymers including, but not limited to collagen, gelatin, chitosan, alginate, hyaluronic acid, dextran; polylactic acid, polyglycolic acid, poly(lactic acid-co-glycolic acid), polycaprolactone, polyanhydride, polyorthoester, polyvinyl alcohol, polyethylene glycol, polyurethane, polyacrylic acid, poly-N-isopropylacrylamide, poly(ethyleneoxide)-poly(propyleneoxide)-poly (ethyleneoxide)copolymer, copolymers thereof and mixtures thereof; said polymer scaffold having an interconnective porous structure with a uniform pore size typically in the range of 10 to 800 μM (preferably 100 to 500 μM) and a porosity in the range 40% to 97% (preferably in the range of 50% to 97% or, more specifically, 70% to 95%); the polymer scaffold being prepared from the said biocompatible polymers using methods well known in the art including, casting/solvent extraction, gas foaming, phase separation, electrospinning, gel spinning, and the like; said polymer scaffold typically having a thickness in the range 0.5 to 3 millimetres; optionally in which said generation method comprises the differentiation and clustering together of chondrocytes to form cell aggregates of appropriate size using hanging drop culture, pellet culture, micromass culture or rotational culture, said cell aggregates being typically in the range 10 to 800 μM, and matched to the pore size of said polymer scaffold material; mixing of the cell aggregates of differentiated chondrocytes with hydrogels in a solution state in a weight ratio in the range of: 1 to 1:100 to form a cell aggregate-hydrogel complex in which said cell aggregates are evenly dispersed, creating a three-dimensional environment physiologically similar to that of natural cartilage (suitable hydrogels including fibrin, gelatin, collagen, hyaluronic acid, agarose, chitosan, polyphosphazine, polyacrylate, polyglactic acid, polyglycolic acid, pluronic acid, alginate, salts and the like, used alone or in mixture form); application in a solution state of the cell aggregates of differentiated chondrocytes evenly dispersed in the hydrogel matrix to said polymer scaffold material, simultaneously filling up the pores thereof and solidifying into a gel state to obtain a cell aggregate-hydrogel-polymer scaffold complex, the gelation method depending upon the type of hydrogel used, the hydrogel creating a three-dimensional environment physiologically similar to that of natural cartilage and the cell aggregate-hydrogel-polymer scaffold complex further improving the efficiency of chondrogenic differentiation, the use of a polymer scaffold enabling the maintenance of high mechanical strength, accurate shaping, flexibility and uniform morphology during the chondrogenic differentiation and the cell aggregate-hydrogel-polymer scaffold complex providing cartilage tissue with high mechanical strength, flexibility, and uniform morphology.

79. The prosthetic knee menisci of claim 62 in which the lubricity of said bearing surfaces of said menisci is enhanced by the generation thereon of a layer of hyaline cartilage, said generation process comprising application to said working surfaces of a fluoridated hydroxyapatite coating on which is deposited a bone-like, microstructured beta-tricalcium phosphate layer to simulate the layer of calcified articular cartilage normally abutting subchondral bone, said fluoridated hydroxyapatite coating being optionally infused into said working surfaces using super-critical carbon dioxide (scCO₂), taking advantage of the solvency and plasticization effects of scCO₂; preparation of a layer of scaffold material and the fusing or otherwise fixing of said scaffold material to said working surfaces; seeding of said scaffold material with articular chondrocytes; cultivation of said chondrocytes in vitro under appropriate conditions.

80. A method of providing prosthetic knee menisci to be implanted in place of deteriorated native menisci to prevent damage to the articular cartilage of the femoral and tibial condyles and, thereby, to arrest the progressive development of osteoarthritis; said method including the provision of prosthetic menisci made as hollow forms which are inflated after implantation by injection of a settable polymer to shape them into congruence with the femoral and tibial condyles; said prosthetic menisci being sized for the femoral and tibial condylar surfaces; having internal reinforcement for strength and durability; being made from materials having elastomeric characteristics similar to those of native menisci; having bearing surfaces treated chemically and/or physically to improve the efficiency of lubrication by synovial fluid and to enhance the wear characteristics of the bearing surfaces; and being restricted in translation within the interarticular space by anchorage of their anterior and posterior horns and by the provision of secondary locating elements.

81. The method of providing prosthetic knee menisci of claim 80 in which, to implant said prosthetic menisci, access is gained to the knee compartment via minimal incisions and separation or displacement of the tendinous and capsular tissue surrounding the joint; the native menisci are removed as required by surgically severing all of their tibial, ligamentary and capsular attachments with careful attention to haemostasis; where only one said native meniscus is removed, the transverse geniculate ligament is severed at an appropriate length and sutured to the base of the anterior cruciate ligament; said prosthetic menisci are selected for planform size and shape from radiographic, ultra-sonic or magnetic resonance-derived images of the condyles; bone is removed as necessary to provide attachment faces for said locating strap anchors; said prosthetic menisci are collapsed by evacuation, folded into compact form, lubricated, loaded into convergent positioning tubes and extruded into position between the femoral and tibial condyles, varus or vagus force being applied as necessary to open the joint; said prosthetic menisci are unfolded and positioned correctly, their horns being extended and their fixing plates secured to the tibia with suitable fastenings; said locating strap anchors with said locating straps are fixed to the tibia; the femoral and tibial condyles are maintained in correct relationship and a settable resin is injected into said prosthetic menisci to inflate them into congruence with said femoral and tibial condyles; the femoral and tibial condyles are maintained in correct relationship until said settable resin has catalysed; finally, the synovial capsule is modified as required to fully enclose the joint, separated tissue is reinstated and the skin incisions are closed.
